SEQUENCE LISTING (9 sequences)

SEQ ID NO: 1 - DNA, 1099 bp, Homo sapiens
ctcatatctttgagataattaaactaattttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcaaagtgcttacagtgcaggtagtgatatgtgcatctactgcagtgaaggcacttgtagcattatggtgacagctgcctcgggaagccaagttgggctttaaagtgcagggcctgctgatgttgagtgctttttgttctaaggtgcatctagtgcagatagtgaagtagattagcatctactgccctaagtgctccttctggcataagaagttatgtattcatccaataattcaagccaagcaagtatataggtgttttaatagtttttgtttgcagtcctctgttagttttgcatagttgcactacaagaagaatgtagttgtgcaaatctatgcaaaactgatggtggcctgctatttccttcaaatgaatgatttttactaattttgtgtacttttattgtgtcgatgtagaatctgcctggtctatctgatgtgacagcttctgtagcactaaagtgcttatagtgcaggtagtgtttagttatctactgcattatgagcacttaaagtactgctagctgtagaactccagcttcggcctgtcgcccaatcaaactgtcctgttactgaacactgttctatggttagttttgcaggtttgcatccagctgtgtgatattctgctgtgcaaatccatgcaaaactgactgtggtagtgaaaagtctgtagaaaagtaagggaaactcaaacccctttctacacaggttgggatcggttgcaatgctgtgtttctgtatggtattgcacttgtcccggcctgttgagtttggtggggattgtgaccagaagattttgaaaattaaatattactgaagatttcgacttccactgttaaatgtacaagatacatgaaatattaaagaaaatgtgtaactttttgtgtaaatacatcttgtcttgttttcattcaaaaac

SEQ ID NO: 2 - DNA, 23 bp, Homo sapiens
caaagtgcttacagtgcaggtag

SEQ ID NO: 3 - DNA, 23 bp, Homo sapiens
taaggtgcatctagtgcagatag

SEQ ID NO: 4 - DNA, 22 bp, Homo sapiens
agttttgcatagttgcactaca

SEQ ID NO: 5 - DNA, 23 bp, Homo sapiens
caaagtgcttacagtgcaggtag

SEQ ID NO: 6 - DNA, 23 bp, Homo sapiens
caaagtgcttacagtgcaggtag

SEQ ID NO: 7 - DNA, 23 bp, Homo sapiens
aggttgggatcggttgcaatgct

SEQ ID NO: 8 - DNA, 2298 bp, Homo sapiens
atggaggaaattaaacctgcctctgcttcttgtgtctcaaaagaaaaacccagtaaggtatcagatctcatcagtcgctttgaaggaggcagctcattatcaaattatagtgatttgaagaaagagtctgctgtgaacctaaatgctcctagaaccccaggaaggcatggattgacaaccacacctcaacaaaaactcctctcccagcacttgccacagaggcagggaaatgatacagataagactcagggtgcacagacttgtgtggccaacggtgtaatggcagcacaaaaccagatggaatgtgaggaggagaaagctgccactcttagctcagatacttctattcaagcttctgaacccttgcttgatatgcacatagtgaatggagaaagagatgaaactgccacagctcctgcatcacccacaacagacagctgtgatggaaatgcttctgacagtagctacaggactccaggcataggcccagtgctccccctagaagaaagaggggcagaaacagaaaccaaggtacaagagagggaaaatggggaaagccctctggaactggagcagctggaccagcaccatgagatgaaggagactaatgagcaaaaacttcacaaaatagccaatgaacttttgcttactgaaagagcttatgtcaaccgacttgacctcttagatcaggtattttattgcaaactgttggaagaagcaaaccgaggctcgtttccagcagagatggtgaataaaatcttttctaatatttcatcaataaatgccttccatagtaaattcctcttgccagagctggagaaacgaatgcaagaatgggaaactactcctagaattggagacatccttcagaaattggcaccattccttaagatgtatggagaatatgtgaaaggatttgataatgcaatggaattggttaaaaacatgacagaacgtattccccagttcaaatcagtggttgaagaaattcagaaacagaaaatctgtgggagcttaactttgcagcatcacatgctagaacctgttcagcggattccccggtatgagatgctccttaaggactatctaaggaaattgcctcctgattccctggactggaatgatgctaaaaaatcacttgaaattatatctacagcagcaagccattctaatagtgcaataaggaaaatggagaacctaaagaaactcttagagatttatgaaatgttgggagaagaagaagacattgtaaacccttcaaatgaactaataaaagaaggacagatcctcaaactagctgctcgaaacacttcagcacaagaacgctaccttttcttattcaacaacatgttgctgtactgtgtgcccaaattcagcttggtaggctctaaattcacagttcgaaccagggttggcattgatggaatgaaaattgtagagactcaaaatgaagaatatccacatactttccaggtgtctgggaaagagagaacactggaactgcaggccagttctgcacaagacaaagaagaatggatcaaggcccttcaagaaaccatcgatgcttttcatcaaaggcatgaaaccttcagaaatgcaattgcaaaggataatgacattcactcagaggtttctactgctgagctagggaaaagagccccaagatggatccgagataatgaagtgacaatgtgtatgaaatgtaaagaacctttcaatgcactgacacgaaggaggcatcattgtcgagcatgtggatatgtggtttgttggaaatgctccgactacaaagctcaacttgaatatgatggtggtaaattgagcaaagtttgtaaagactgttatcaaatcataagtggattcacagacagtgaagaaaagaaaagaaaaggaattttagagattgaatcagcagaagtatctggaaacagtgtggtgtgcagctttcttcagtatatggagaagtcaaaaccttggcagaaagcttggtgtgtgatccccaagcaagaccctcttgtgctgtacatgtatggtgccccccaggacgtcagagcccaggccaccattccacttctgggctatgtggtggatgaaatgccaaggagcgcagacctgccacacagtttcaaactgacccagtctaagtccgtgcacagctttgctgcagacagtgaggaactgaagcagaagtggctgaaagtcatccttttagctgtcacaggtgagacaccaggtggtccaaatgagcatccagccaccttggatgatcatcctgaacctaagaaaaaatcagaatgc

SEQ ID NO: 9 - protein, 766 aa, Homo sapiens [sequence omitted]

(19) United States
(12) Patent Application Publication
(10) Pub. No.: US 2015/0238519 A1
(43) Pub. Date: Aug. 27, 2015

(54) THERAPY FOR DRUG RESISTANT CANCER

(71) Applicants: Ratna Chakrabarti, Winter Springs, FL (US); Richard Ottman, Orlando, FL (US)

(72) Inventors: Ratna Chakrabarti, Winter Springs, FL (US); Richard Ottman, Orlando, FL (US)

(73) Assignee: University of Central Florida Research Foundation, Inc., Orlando, FL (US)

(21) Appl. No.: 14/626,852

(22) Filed: Feb. 19, 2015

Related U.S. Application Data
(60) Provisional application No. 61/941,877, filed on Feb. 19, 2014.

(51) Int. Cl. A61K 31/7105; C07K 16/18; A61K 31/713

(52) U.S. Cl. CPC A61K 31/7105; A61K 31/713; C07K 16/18; C07K 2317/76

(57) ABSTRACT

Disclosed herein are compositions and methods for treating cancer involving the targeting of FGD4. Alternatively, methods and compositions involve the administration of certain miRNA sequences. The administration is effective to reduce the conversion of cancer cells to an aggressive phenotype.

### STATEMENT OF GOVERNMENT SUPPORT

The invention was made with government support under grant no. W*1XWH-11-1-0563 awarded by the U.S. Department of Defense. The government has certain rights in the invention

### INTRODUCTION

Androgen blockade therapy has become the mainstay for advanced prostate cancer. However, prolonged androgen blockade leads to outgrowth of androgen independent (AI) cells and the development of castration resistant prostate cancer (CRPC). The transition to androgen independence can occur through several adaptive mechanisms and usually results in the acquisition of a more aggressive phenotype, compared to their androgen sensitive progenitors.

### BRIEF DESCRIPTION OF THE DRAWINGS

FIG. 1. A. Fold change in expression of mir-17, -18a, -18b, -20a and -106a as determined by qRT-PCR. Data is the average of 3 replicates FIG. 1. B. Hierarchical clustering of genome-wide miRNA expression profile in LNCaP cells in four treatment conditions during progression of CDX resistance. Increased expression: red, decreased expression: green. FIG. 1. C. Profiling summary information. After validation, a panel of 43 miRNA was identified that were significantly altered.

FIG. 2. Expression of FGD4 in untreated LNCap-104R1, LNCaP-104S and LNCaP-104S cells treated with CDX and androgen withdrawal. FIG. 2 A: Western blot of the whole cell extracts. FIG. 2 B: Densitometric analysis of the expression. Data represents the average of 3 separate analyses.

FIG. 3. A. Graphical representation of Frabin expression in AI, metastatic, hi, med, low Gleason, hi and low PIN and BPH tissues. Values above the bar represent the percentage of tissues with staining intensity above the threshold. FIG. 3. B. Representative images of expression of FGD4 in prostate tumors, PIN and BPH. Inset: Enlarged representative sections.

FIG. 4. Expression of mir-17, -20a and -106a in prostate tumors. Data shows fold change in expression compared to matched uninvolved tissues.

FIG. 5. Immunofluorescence analysis of Frabin and mir-17-92 cluster. GFP is from the expression of mir-17-92 cluster. Red fluorescence: Frabin, DAPI: Nuclear stain, Merge is with DIC+Frabin and DAPI. Scalr bar: 10 μm

FIG. 6. Effect of DTX treatment (10 nM for 48 hrs.) on mir-17-92 cluster expressΔing PC3 cells. Upper panel: Transfected cells (GFP) showing loss of Frabin and multinucleation. Lower panel: Vector transfected cell. Arrow: untransfected cell. Scale bar: 10 μm

FIG. 7A. Expression of miRNAs in prostate tissues. miR-17, -20a and -106a; Data shows fold change in expression (ΔΔCT values) compared to matched uninvolved tissues. Frequency of down regulation: miR-17 (76%), miR-20a (62%), miR-106a (86%), miR-18a(33%), miR-19a (67%) and miR-92a (62%).

FIG. 7B.: Expression of MiRNAs in prostate tissues. miR-18a, -19a and -92a. Data shows fold change in expression (ΔΔCT values) compared to matched uninvolved tissues. Frequency of down regulation: miR-17 (76%), miR-20a (62%), miR-106a (86%), miR-18a (33%), miR-19a (67%) and miR-92a (62%).

FIG. 8. Tumor growth in xenografts (NSG) of M12 cells (1×10⁶) expressing miR-17-92 cluster or Scr RNA. Data shows mean tumor volume±SD of 4 animals in each group.

FIG. 9. Tumor growth in xenografts (NSG) of PC-3 cells (1×10⁶) expressing miR-17-92 cluster (>1.6-1.8-fold higher miR-17 and -20a expression as detected by qRT-PCR) or Scr RNA. Data shows mean tumor volume±SD of 2 animals in each group.

### DETAILED DESCRIPTION

MicroRNAs (miRNAs) are small noncoding RNAs that regulate protein expression through translational inhibition and play important roles in regulation of gene expression for cancer progression, metastasis, and resistance to therapeutic strategies. A specific cluster of five microRNAs has been identified that is down regulated during transition of androgen sensitive (AS) LNCaP prostate cancer cells to AI and Casodex (CDX) (androgen receptor antagonist) resistant cells. Using genome-wide expression profiling of miRNAs from AS and AI sublines of LNCaP cells, it was found that a subset of miRNAs that are significantly deregulated in these cells. This cluster is one of the groups of miRNAs that are down regulated as the cancer cells progressed towards androgen blockade therapy (ADT) resistance. More than 4 to 24-fold down regulation of these miRNAs were noted in CDX resistant cells compared to AS LNCaP cells.

The expression status of this microRNA cluster was monitored in patient tumor tissues, which showed down regulation of these miRNAs in 64-82% of the tissues tested. Target prediction database searches identified a specific protein as one of the targets of this cluster. Western blot analysis of treated cell lysates confirmed increased expression of this protein in AI and CDX resistant LNCaP cells.

Analysis in tissue microarray (267 cores) showed a significant up regulation of this target protein in advanced prostate cancer tissues including AI specimens. More than 90% of the AI tissues and 88% of tissues with 8-10 Gleason scores showed a median staining intensity between 2-3-fold higher compared to BPH tissues. Ectopic expression of this miRNA clusters in AI PC3 cells down regulated the target protein expression and improved sensitivity of these cells to docetaxel (DTX) treatments. Based on the findings disclosed herein, certain embodiments of the invention involve the use of miRNAs in this cluster as therapy for AI and aggressive cancer. Also, other embodiments involve the targeting of miRNA intracellular targets for cancer therapy.

Disclosed are methods of ameliorating, preventing, delaying the onset, improving or treating an unwanted condition, disease or symptom of a patient in need. A patient in need is typically one who has cancer, and in particular an aggressive prostate cancer. In particular, certain method embodiments involve the delivery of certain interfering molecules or inhibitor molecules that mimic an miRNA from the miRNA-17-92a cluster. In addition, other method embodiments involve the target expression of FGD4. The method embodiments provide a cancer therapy that reduces conversion of cancer cells to an aggressive phenotype and/or reduces aggressiveness of cancer.

As used herein, “aggressive” or “aggressiveness” as it refers to cancer is intended to mean resistant or insensitive to chemotherapy, increased malignancy compared to an initial phenotype, and/or increased metastasis compared to an initial phenotype.

In a particular embodiment, provided is a method for treating cancer in a subject, comprising administering to the subject a therapeutically effective amount of a composition that inhibits the expression or action of FGD4 in the subject. In a typical embodiment, the cancer is prostate cancer. In a more specific embodiment, the method involves co-administration with convention chemotherapies.

In another embodiment, provided is a method for treating cancer that involves the administration of miRNA molecules according to SEQ ID NOs. 1, 2, 3, 4, 5, 6, and/or 7.

As used herein and in the appended claims, the singular forms “a”, “an”, and “the” include plural reference unless the context clearly dictates otherwise. Thus, for example, reference to an “compound” is a reference to one or more compounds and equivalents thereof known to those skilled in the art, and so forth.

As used herein, the term “about” means plus or minus 10% of the numerical value of the number with which it is being used. Therefore, about 50% means in the range of 45%-55%.

“Administering” when used in conjunction with a therapeutic means to administer a therapeutic to a patient whereby the therapeutic positively impacts the tissue to which it is targeted. The compounds described herein can be administered either alone or in combination (concurrently or serially) with other pharmaceuticals. For example, the compounds can be administered in combination with other antioxidants or agents known to treat the target condition. In some embodiments, the compounds described herein can also be administered in combination with (i.e., as a combined formulation or as separate formulations) with antibiotics.

The terms “animal,” “patient,” or “subject” are used interchangeably, and include, but are not limited to, humans and non-human vertebrates such as wild, domestic and farm animals. Typically, the term refers to humans.

By “pharmaceutically acceptable”, it is meant the carrier, diluent or excipient must be compatible with the other ingredients of the formulation and not deleterious to the recipient thereof.

As used herein, the term “therapeutic” means an agent utilized to discourage, combat, ameliorate, prevent or improve an unwanted condition, disease or symptom of a patient.

A “therapeutically effective amount” or “effective amount” of a composition is a predetermined amount calculated to achieve the desired effect, i.e., to ameliorate, treat, prevent or improve an unwanted condition, disease or symptom of a patient. In a specific example, a therapeutically effective amount is one that reduces the aggressiveness of cancer, or reduces the androgen blockade therapy insensitivity. The activity contemplated by the present methods includes both therapeutic and/or prophylactic treatment, as appropriate. The specific dose of the compounds or the compounds administered according to this invention to obtain therapeutic and/or prophylactic effects will, of course, be determined by the particular circumstances surrounding the case, including, for example, the compounds administered, the route of administration, and the condition being treated. The effective amount administered may be determined by a physician in the light of the relevant circumstances including the condition to be treated, the choice of compounds to be administered, and the chosen route of administration. A therapeutically effective amount of the compound/compound of this invention is typically an amount such that when it is administered in a physiologically tolerable excipient composition, it is sufficient to achieve an effective systemic concentration or local concentration in the target tissue.

Generally speaking, the term “tissue” refers to any aggregation of similarly specialized cells which are united in the performance of a particular function.

In a specific embodiment, disclosed is an lentivirus-mediated overexpression or knockdown system and an effective method for the genetic intervention of FGD4, specifically in prostate cancer cells in vivo.

Another embodiment relates to a cell-based assay for identification of agents that reduce aggressiveness of cancer cells. The assay includes a. contacting a population of androgen insensitive prostate cancer cells with a test agent, b. determining level of agressiveness in the test population, and c. selecting the test agent if the level of agressiveness in the test population is significantly higher than the level in a control population.

### Target Sequences

Target Sequences include an FGD4 sequence, such as that of SEQ ID NO. 8, and a fragment thereof. In should be recognized that reference to FGD4 target sequences also includes the targeting of RNA transcripts of the FGD4 gene. In particular, target sequences include those sequences of FGD4 that are also targeted by any of SEQ ID NOs 2-7.

### Compounds

Compounds of the present disclosure pertain to those able to modulate expression, RNA processing, translation or activity of FGD4 or components thereof. Such compounds are also referred to herein as agent of interest (AOI) compounds. The AOI compounds may be a RNA interfering molecule, antibody, antisense molecule, PMO, ribozyme or small molecule. Compounds or AOI compounds as used herein include not only refer to the inhibitor but also refer to a delivery vehicle for providing the inhibitor. For example, reference to AOI compound or compound may refer to RNA interfering molecule or to a viral vector or delivery vector including a sequence to express the RNA interfering molecule.

RNA interference (RNAi) is a process by which double-stranded RNA (dsRNA) is used to silence gene expression. While not wanting to be bound by theory, RNAi begins with the cleavage of longer dsRNAs into small interfering RNAs (siRNAs) by an RNaseIII-like enzyme, dicer. SiRNAs are dsRNAs that are usually about 19 to 28 nucleotides, or 20 to 25 nucleotides, or 21 to 22 nucleotides in length and often contain 2-nucleotide 3′ overhangs, and 5′ phosphate and 3′ hydroxyl termini. One strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. Therefore, the siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of an siRNA can be incorporated into RISC and function as a guide strand. However, siRNA design (e.g., decreased siRNA duplex stability at the 5′ end of the desired guide strand) can favor incorporation of the desired guide strand into RISC.

The antisense strand of an siRNA is the active guiding agent of the siRNA in that the antisense strand is incorporated into RISC, thus allowing RISC to identify target mRNAs with at least partial complementarity to the antisense siRNA strand for cleavage or translational repression. RISC-related cleavage of mRNAs having a sequence at least partially complementary to the guide strand leads to a decrease in the steady state level of that mRNA and of the corresponding protein encoded by this mRNA. Alternatively, RISC can also decrease expression of the corresponding protein via translational repression without cleavage of the target mRNA.

The term “siRNA” as used herein refers to a double-stranded interfering RNA unless otherwise noted. Typically, an siRNA of the invention is a double-stranded nucleic acid molecule comprising two nucleotide strands, each strand having about 19 to about 28 nucleotides (i.e. about 19, 20, 21, 22, 23, 24, 25, 26, 27, or 28 nucleotides). The phrase “interfering RNA having a length of 19 to 49 nucleotides” when referring to a double-stranded interfering RNA means that the antisense and sense strands independently have a length of about 19 to about 49 nucleotides, including interfering RNA molecules where the sense and antisense strands are connected by a linker molecule.

In addition to siRNA molecules, other interfering RNA molecules and RNA-like molecules can interact with RISC and silence gene expression. Examples of other interfering RNA molecules that can interact with RISC include short hairpin RNAs (shRNAs), single-stranded siRNAs, microRNAs (miRNAs), and dicer-substrate 27-mer duplexes. Examples of RNA-like molecules that can interact with RISC include siRNA, single-stranded siRNA, microRNA, and shRNA molecules containing one or more chemically modified nucleotides, one or more non-nucleotides, one or more deoxyribonucleotides, and/or one or more non-phosphodiester linkages. All RNA or RNA-like molecules that can interact with RISC and participate in RISC-related changes in gene expression are referred to herein as “interfering RNAs” or “interfering RNA molecules.” SiRNAs, single-stranded siRNAs, shRNAs, miRNAs, and dicer-substrate 27-mer duplexes are, therefore, subsets of “interfering RNAs” or “interfering RNA molecules.”

Single-stranded interfering RNA has been found to effect mRNA silencing, albeit less efficiently than double-stranded RNA. Therefore, embodiments of the present invention also provide for administration of a single-stranded interfering RNA that has a region of at least near-perfect contiguous complementarity with a portion of the FGD4 mRNA. The single-stranded interfering RNA has a length of about 19 to about 49 nucleotides as for the double-stranded interfering RNA cited above. The single-stranded interfering RNA has a 5′ phosphate or is phosphorylated in situ or in vivo at the 5′ position. The term “5′ phosphorylated” is used to describe, for example, polynucleotides or oligonucleotides having a phosphate group attached via ester linkage to the C5 hydroxyl of the sugar (e.g., ribose, deoxyribose, or an analog of same) at the 5′ end of the polynucleotide or oligonucleotide.

Single-stranded interfering RNAs can be synthesized chemically or by in vitro transcription or expressed endogenously from vectors or expression cassettes as described herein in reference to double-stranded interfering RNAs. 5′ Phosphate groups may be added via a kinase, or a 5′ phosphate may be the result of nuclease cleavage of an RNA. A hairpin interfering RNA is a single molecule (e.g., a single oligonucleotide chain) that comprises both the sense and antisense strands of an interfering RNA in a stem-loop or hairpin structure (e.g., a shRNA). For example, shRNAs can be expressed from DNA vectors in which the DNA oligonucleotides encoding a sense interfering RNA strand are linked to the DNA oligonucleotides encoding the reverse complementary antisense interfering RNA strand by a short spacer. If needed for the chosen expression vector, 3′ terminal T's and nucleotides forming restriction sites may be added. The resulting RNA transcript folds back onto itself to form a stem-loop structure.

Nucleic acid sequences cited herein are written in a 5′ to 3′ direction unless indicated otherwise. The term “nucleic acid,” as used herein, refers to either DNA or RNA or a modified form thereof comprising the purine or pyrimidine bases present in DNA (adenine “A,” cytosine “C,” guanine “0,” thymine “T”) or in RNA (adenine “A,” cytosine “C,” guanine “G,” uracil “U”). Interfering RNAs provided herein may comprise “T” bases, particularly at 3′ ends, even though “T” bases do not naturally occur in RNA. “Nucleic acid” includes the terms “oligonucleotide” and “polynucleotide” and can refer to a single-stranded molecule or a double-stranded molecule. A double-stranded molecule is formed by Watson-Crick base pairing between A and T bases, C and G bases, and between A and U bases. The strands of a double-stranded molecule may have partial, substantial or full complementarity to each other and will form a duplex hybrid, the strength of bonding of which is dependent upon the nature and degree of complementarity of the sequence of bases.

In certain embodiments, interfering RNA target sequences (e.g., si RNA target sequences) within a target mRNA sequence are selected using available design tools. Interfering RNAs corresponding to a FGD4 target sequence are then tested in vitro by transfection of cells expressing the target mRNA followed by assessment of knockdown as described herein. The interfering RNAs can be further evaluated in vivo using animal models as described herein.

Techniques for selecting target sequences for si RNAs are provided, for example, by Tuschl, T. et al., “The siRNA User Guide,” revised May 6, 2004, available on the Rockefeller University web site; by Technical Bulletin #506, “siRNA Design Guidelines,” Ambion Inc. at Ambion's web site; and by other web-based design tools at, for example, the Invitrogen, Dharmacon, Integrated DNA Technologies, Genscript, or Proligo web sites. Initial search parameters can include G/C contents between 35% and 55% and siRNA lengths between 19 and 27 nucleotides. The target sequence may be located in the coding region or in the 5′ or 3′ untranslated regions of the mRNA. The target sequences can be used to derive interfering RNA molecules, such as those described herein.

In certain embodiments, silencing of FGD4 may be based on SEQ ID NOS 1-7.

Many of the embodiments of the subject invention make reference to particular methods of inhibiting or disruption of genetic expression. Based on the teachings herein, methods of inhibiting expression include but are not limited to siRNA; ribozyme(s); antibody(ies); antisense/oligonucleotide(s); morpholino oligomers; microRNA; or shRNA that target expression of the FGD4. The subject invention is not to be limited to any of the particular related methods described. One such method includes siRNA (small interfering/short interfering/silencing RNA). SiRNA most often is involved in the RNA interference pathway where it interferes with the expression of a specific gene. In addition to its role in the RNA interference pathway, siRNA also act in RNA interference-related pathways, e.g., as an antiviral mechanism or in shaping the chromatin structure of a genome.

Another method by which to inhibit expression and to inhibit the expression of FGD4 in particular is shRNA. ShRNA (short hairpin or small hairpin RNA) refers to a sequence of RNA that makes a tight hairpin turn and is used to silence gene expression via RNA interference. It uses a vector introduced into cells and a U6 or H1 promoter to ensure that the shRNA is always expressed. The shRNA hairpin structure is cleaved by cellular machinery into siRNA which is then bound to the RNA-induced silencing complex. This complex binds to and cleaves mRNAs which match the siRNA that is bound to it.

FGD4 can also be blocked by subjecting cells to an antibody specific to frabin. An antisense nucleotide may also be used to block or inhibit expression, in particular, the expression of FGD4. Expression may also be inhibited with the use of a morpholino oligomer or phosphorodiamidate morpholino oligomer (PMO). PMOs are an antisense technology used to block access of other molecules to specific sequences within nucleic acid. PMOs are often used as a research tool for reverse genetics, and function by knocking down gene function. This is achieved by preventing cells from making a targeted protein or by modifying splicing of pre-mRNA. One embodiment of the subject disclosure pertains to a method of treating neurons under oxidative stress by expressing an RNA interfering molecule, antisense molecule or PMO in a subject in need thereof.

### Antibodies

Agents that reduce the biological activity of a FGD4 protein (Frabin) include antibodies (including portions or fragments or variants of antibody fragments or variants of antibodies) that have specific binding affinity for the intended FGD4 protein, thereby interfering with its biological activity. These antibodies recognize an epitope in a target protein or biologically active fragment thereof, namely frabin.

An “antibody” refers to an intact immunoglobulin or to an antigen-binding portion (fragment) thereof that competes with the intact antibody for specific binding, and is meant to include bioactive antibody fragments. Therapeutically useful antibodies in treating or preventing an enumerated disease or changing a phenotype as described include any antibody to any frabin protein or analog, ortholog or variant thereof, that binds to or reduces the biological activity of frabin.

Once produced, antibodies or fragments thereof can be tested for recognition of the target polypeptide by standard immunoassay methods including, for example, enzyme-linked immunosorbent assay (ELISA) or radioimmunoassay assay (RIA). See, Short Protocols in Molecular Biology eds. Ausubel et al., Green Publishing Associates and John Wiley & Sons (1992).

The term “epitope” refers to an antigenic determinant on an antigen to which an antibody binds. Epitopes usually consist of chemically active surface groupings of molecules such as amino acids or sugar side chains, and typically have specific three-dimensional structural characteristics, as well as specific charge characteristics. Epitopes generally have at least five contiguous amino acids. The terms “antibody” and “antibodies” include polyclonal antibodies, monoclonal antibodies, humanized or chimeric antibodies, single chain Fv antibody fragments, Fab fragments, and F(ab′)₂ fragments. Polyclonal antibodies are heterogeneous populations of antibody molecules that are specific for a particular antigen, while monoclonal antibodies are homogeneous populations of antibodies to a particular epitope contained within an antigen. Monoclonal antibodies are particularly useful in the present invention.

Antibody fragments that have specific binding affinity for the polypeptide of interest can be generated by known techniques. Such antibody fragments include, but are not limited to, F(ab′)₂ fragments that can be produced by pepsin digestion of an antibody molecule, and Fab fragments that can be generated by reducing the disulfide bridges of F(ab′)₂ fragments. Alternatively, Fab expression libraries can be constructed. See, for example, Huse et al. (1989) Science 246:1275-1281. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778.

An “isolated antibody” is an antibody that (1) is not associated with naturally-associated components, including other naturally-associated antibodies, that accompany it in its native state, (2) is free of other proteins from the same species, (21) is expressed by a cell from a different species, or (4) does not occur in nature.

The term “human antibody” includes all antibodies that have one or more variable and constant regions derived from human immunoglobulin sequences. In a preferred embodiment, all of the variable and constant domains are derived from human immunoglobulin sequences (a fully human antibody). These antibodies may be prepared in a variety of ways, as described below.

A humanized antibody is an antibody that is derived from a non-human species, in which certain amino acids in the framework and constant domains of the heavy and light chains have been mutated so as to avoid or abrogate an immune response in humans. Alternatively, a humanized antibody may be produced by fusing the constant domains from a human antibody to the variable domains of a non-human species. Examples of how to make humanized antibodies may be found in U.S. Pat. Nos. 6,054,297, 5,886,152 and 5,877,293, incorporated herein by reference.

The term “chimeric antibody” refers to an antibody that contains one or more regions from one antibody and one or more regions from one or more other antibodies.

Fragments, portions or analogs of antibodies can be readily prepared by those of ordinary skill in the art following the teachings of this specification. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991).

Antisense Nucleotides and siRNA

Other embodiments of the present invention are directed to the use of antisense nucleic acids to reduce or inhibit expression of FGD4. Based on these known sequences of the targeted FGD4 protein and genes encoding them, antisense DNA or RNA that are sufficiently complementary to the respective gene or mRNA to turn off or reduce expression can be readily designed and engineered, using methods known in the art. In a specific embodiment of the invention, antisense or siRNA molecules for use in the present invention are those that bind under stringent conditions to the targeted mRNA or targeted gene encoding FGD4 protein. The antisense compounds of the invention are synthesized in vitro and do not include antisense compositions of biological origin.

Methods of making antisense nucleic acids are well known in the art. Further provided are methods of reducing the expression of FGD4 and mRNA in cells (e.g. cancer cells) by contacting the cells in situ or contacting isolated enriched populations of the cells or tissue explants in culture that comprise the cells with one or more of the antisense compounds or compositions of the invention. As used herein, the terms “target nucleic acid” encompass DNA encoding FGD4 protein and RNA (including pre-mRNA and mRNA) transcribed from such DNA. The specific hybridization of a nucleic acid oligomeric compound with its target nucleic acid interferes with the normal function of the target nucleic acid. This modulation of function of a target nucleic acid by compounds which specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, and catalytic activity which may be engaged in or facilitated by the RNA. The overall effect of such interference with target nucleic acid function is modulating or reducing the expression of the protein encoded by the DNA or RNA. In the context of the present invention, “modulation” means reducing or inhibiting in the expression of the gene or mRNA FGD4 or Frabin.

The targeting process includes determination of a site or sites within the target DNA or RNA encoding the FGD4 protein for the antisense interaction to occur such that the desired inhibitory effect is achieved. Within the context of the present invention, a preferred intragenic site is the region encompassing the translation initiation or termination codon of the open reading frame (ORF) of the mRNA for the targeted proteins. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine in eukaryotes. It is also known in the art that eukaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene. Routine experimentation will determine the optimal sequence of the antisense or siRNA

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

Once one or more target sites have been identified, nucleic acids are chosen which are sufficiently complementary to the target; meaning that the nucleic acids will hybridize sufficiently well and with sufficient specificity, to give the desired effect of inhibiting gene expression and transcription or mRNA translation.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of a nucleic acid is capable of hydrogen bonding with a nucleotide at the same position of a DNA or RNA molecule, then the nucleic acid and the DNA or RNA are considered to be complementary to each other at that position. The nucleic acid and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the nucleic acid and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of function, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

While antisense nucleic acids are a preferred form of antisense compound, the present invention comprehends other oligomeric antisense compounds, including but not limited to oligonucleotide mimetics. The antisense compounds in accordance with this invention preferably comprise from about 8 to about 50 nucleobases (i.e., from about 8 to about 50 linked nucleosides). Particularly preferred antisense compounds are antisense nucleic acids comprising from about 12 to about 30 nucleobases. Antisense compounds include ribozymes, external guide sequence (EGS) nucleic acids (oligozymes), and other short catalytic RNAs or catalytic nucleic acids which hybridize to the target nucleic acid and modulate its expression. Nucleic acids in the context of this invention include “oligonucleotides,” which refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

Antisense nucleic acids have been employed as therapeutic moieties in the treatment of disease states in animals and man. Antisense nucleic acid drugs, including ribozymes, have been safely and effectively administered to humans and numerous clinical trials are presently underway. It is thus established that nucleic acids can be useful therapeutic modalities that can be configured to be useful in treatment regimes for treatment of cells, tissues and animals, especially humans, for example to down-regulate expression of FGD4.

The antisense and siRNA compounds referred to herein can be utilized for diagnostics, therapeutics, and prophylaxis and as research reagents and kits. For therapeutics, an animal, preferably a human, suspected of having a disease or disorder such as cancer, (e.g. androgen insensitive prostate cancer), which can be treated by reducing the expression of FGD4, is treated by administering antisense compounds in accordance with this disclosure. The compounds described herein can be utilized in pharmaceutical compositions by adding an effective amount of an antisense compound to a suitable pharmaceutically acceptable diluent or carrier.

Alternatively, antisense nucleic acid molecules, or other RNA interfering molecules, can be modified to target selected cells and then administered systemically. For example, for systemic administration, antisense molecules, miRNA, siRNA, or shRNA, can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred. In a specific embodiment, the antibody complexed with the FGD4 inhibitor targets the phospholipase A2 receptor or androgen receptor (AR).

### Vectors

In some embodiments, viral vectors are used to transfect cells with an AOI. In a particular embodiment, lentivirus or adeno-associated viral vectors are used. Other vectors of the invention used in vitro, in vivo, and ex vivo include viral vectors, such as other retroviruses, herpes viruses, alphavirus, adenovirus, vaccinia virus, papillomavirus, or Epstein Barr virus (EBV).

Methods for constructing and using viral vectors are known in the art (see, e.g., Miller and Rosman, BioTechniques 1992, 7:980-990). In accordance with the present invention there may be employed conventional molecular biology, microbiology, and recombinant DNA techniques within the skill of the art. Such techniques are well-known and are explained fully in the literature. See, e.g., Sambrook, Fritsch and Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors) and Origene (Rockville, Md.).

In certain embodiments, the viral vectors of the invention are replication defective, that is, they are unable to replicate autonomously in the target cell. Preferably, the replication defective virus is a minimal virus, i.e., it retains only the sequences of its genome which are necessary for target cell recognition and encapsidating the viral genome. Replication defective virus is not infective after introduction into a cell. Use of replication defective viral vectors allows for administration to cells in a specific, localized area, without concern that the vector can infect other cells. Thus, a specific tissue can be specifically targeted. Examples of particular vectors include, but are not limited to, defective herpes virus vectors (see, e.g., Kaplitt et al., Molec. Cell. Neurosci. 1991, 2:320-330; Patent Publication RD 371005 A; PCT Publications No. WO 94/21807 and WO 92/05263), defective adenovirus vectors (see, e.g., Stratford-Perricaudet et al., J. Clin. Invest. 1992, 90:626-630; La Salle et al., Science 1993, 259:988-990; PCT Publications No. WO 94/26914, WO 95/02697, WO 94/28938, WO 94/28152, WO 94/12649, WO 95/02697, and WO 96/22378), and defective adeno-associated virus vectors (Samulski et al., J. Virol. 1987, 61:3096-3101; Samulski et al., J. Virol. 1989, 63:3822-3828; Lebkowski et al., Mol. Cell. Biol. 1988, 8:3988-3996; PCT Publications No. WO 91/18088 and WO 93/09239; U.S. Pat. Nos. 4,797,368 and 5,139,941; European Publication No. EP 488 528).

Adeno-Associated Virus-Based Vectors.

The adeno-associated viruses (AAV) are DNA viruses of relatively small size which can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see PCT Publications No. WO 91/18088 and WO 93/09239; U.S. Pat. Nos. 4,797,368 and 5,139,941; EP Publication No. 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (e.g., an adenovirus). The AAV recombinants which are produced are then purified by standard techniques.

Adenovirus-Based Vectors.

Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5) or adenoviruses of animal origin (see PCT Publication No. WO94/26914). Those adenoviruses of animal origin which can be used within the scope of the present invention include adenoviruses of canine, bovine, murine (e.g., Mav1 [Beard et al., Virology, 1990, 75:81]), ovine, porcine, avian, and simian (e.g., SAV) origin. Preferably, the adenovirus of animal origin is a canine adenovirus, more preferably a CAV2 adenovirus (e.g., Manhattan or A26/61 strain [ATCC Accession No. VR-800]). Various replication defective adenovirus and minimum adenovirus vectors have been described (PCT Publications No. WO94/26914, WO95/02697, WO94/28938, WO94/28152, WO94/12649, WO95/02697, WO96/22378). The replication defective recombinant adenoviruses according to the invention can be prepared by any technique known to the person skilled in the art (Levrero et al., Gene, 1991, 101:195; EP Publication No. 185 573; Graham, EMBO J., 1984, 3:2917; Graham et al., J. Gen. Virol., 1977, 36:59). Recombinant adenoviruses are recovered and purified using standard molecular biological techniques, which are well known to one of ordinary skill in the art.

Retroviral Vectors.

In another embodiment, the invention provides retroviral vectors, e.g., as described in Mann et al., Cell 1983, 33:153; U.S. Pat. Nos. 4,650,764, 4,980,289, 5,124,263, and 5,399,346; Markowitz et al., J. Virol. 1988, 62:1120; EP Publications No. 453 242 and 178 220; Bernstein et al. Genet. Eng. 1985, 7:235; McCormick, BioTechnology 1985, 3:689; and Kuo et al., 1993, Blood, 82:845. The retroviruses are integrating viruses which infect dividing cells. The retrovirus genome includes two LTRs, an encapsidation sequence and three coding regions (gag, pol and env). Replication defective non-infectious retroviral vectors are manipulated to destroy the viral packaging signal, but retain the structural genes required to package the co-introduced virus engineered to contain the heterologous gene and the packaging signals. Thus, in recombinant replication defective retroviral vectors, the gag, pol and env genes are generally deleted, in whole or in part, and replaced with a heterologous nucleic acid sequence of interest. These vectors can be constructed from different types of retroviruses, such as HIV (human immuno-deficiency virus), MoMuLV (murine Moloney leukaemia virus), MSV (murine Moloney sarcoma virus), HaSV (Harvey sarcoma virus), SNV (spleen necrosis virus), RSV (Rous sarcoma virus), and Friend virus. Suitable packaging cell lines have been described in the prior art, in particular, the cell line PA317 (U.S. Pat. No. 4,861,719); the PsiCRIP cell line (PCT Publication No. WO 90/02806) and the GP+envAm-12 cell line (PCT Publication No. WO 89/07150). In addition, recombinant retroviral vectors can contain modifications within the LTRs for suppressing transcriptional activity as well as extensive encapsidation sequences which may include a part of the gag gene (Bender et al., J. Virol. 1987, 61:1639). Recombinant retroviral vectors are purified by standard techniques known to those having ordinary skill in the art.

Retrovirus vectors can also be introduced by DNA viruses, which permits one cycle of retroviral replication and amplifies transfection efficiency (see PCT Publications No. WO 95/22617, WO 95/26411, WO 96/39036, WO 97/19182).

In a specific embodiment of the invention, lentiviral vectors can be used as agents for the direct delivery and sustained expression of a transgene in several tissue types, including prostate, brain, retina, muscle, liver, and blood. This subtype of retroviral vectors can efficiently transduce dividing and nondividing cells in these tissues, and maintain long-term expression of the gene of interest (for a review, see, Naldini, Curr. Opin. Biotechnol. 1998, 9:457-63; Zufferey, et al., J. Virol. 1998, 72:9873-E) 80). Lentiviral packaging cell lines are available and known generally in the art (see, e.g., Kafri, et al., J. Virol., 1999, 73: 576-584).

Non-Viral Vectors.

In another embodiment, the invention provides non-viral vectors that can be introduced in vivo, provided that these vectors contain a targeting peptide, protein, antibody, etc. that specifically binds HALR. For example, compositions of synthetic cationic lipids, which can be used to prepare liposomes for in vivo transfection of a vector carrying an anti-tumor therapeutic gene, are described in Feigner et. al., Proc. Natl. Acad. Sci. USA 1987, 84:7413-7417; Feigner and Ringold, Science 1989, 337:387-388; Mackey, et al., Proc. Natl. Acad. Sci. USA 1988, 85:8027-8031; and Ulmer et al, Science 1993, 259:1745-1748. Useful lipid compounds and compositions for transfer of nucleic acids are described, e.g., in PCT Publications No. WO 95/18863 and WO96/17823, and in U.S. Pat. No. 5,459,127. Targeting peptides, e.g., laminin or HALR-binding laminin peptides, and proteins such as anti-HALR antibodies, or non-peptide molecules can be coupled to liposomes covalently (e.g., by conjugation of the peptide to a phospholipid or cholesterol; see also Mackey et al., supra) or non-covalently (e.g., by insertion via a membrane binding domain or moiety into the bilayer membrane).

Alphaviruses are well known in the art, and include without limitation Equine Encephalitis viruses, Semliki Forest virus and related species, Sindbis virus, and recombinant or ungrouped species (see Strauss and Strauss, Microbiol. Rev. 1994, 58:491-562, Table 1, p. 493).

As used herein the term “replication deficient virus” has its ordinary meaning, i.e., a virus that is propagation incompetent as a result of modifications to its genome. Thus, once such recombinant virus infects a cell, the only course it can follow is to express any viral and heterologous protein contained in its genome. In a specific embodiment, the replication defective vectors of the invention may contain genes encoding nonstructural proteins, and are self-sufficient for RNA transcription and gene expression. However, these vectors lack genes encoding structural proteins, so that a helper genome is needed to allow them to be packaged into infectious particles. In addition to providing therapeutically safe vectors, the removal of the structural proteins increases the capacity of these vectors to incorporate more than 6 kb of heterologous sequences. In another embodiment, propagation incompetence of the adenovirus vectors of the invention is achieved indirectly, e.g., by removing the packaging signal which allows the structural proteins to be packaged in virions being released from the packaging cell line. As discussed above, viral vectors used to transfect cells and express FGD4 inhibitors may be used, and in a specific embodiment, the viral vectors involve a replication deficient virus.

### Other Delivery Vehicles

Many nonviral techniques for the delivery of a nucleic acid sequence into a cell can be used, including direct naked DNA uptake (e.g., Wolff et al., Science 247: 1465-1468, 1990), receptor-mediated DNA uptake, e.g., using DNA coupled to asialoorosomucoid which is taken up by the asialoglycoprotein receptor in the liver (Wu and Wu, J. Biol. Chem. 262: 4429-4432, 1987; Wu et al., J. Biol. Chem. 266: 14338-14342, 1991), and liposome-mediated delivery (e.g., Kaneda et al., Expt. Cell Res. 173: 56-69, 1987; Kaneda et al., Science 243: 375-378, 1989; Zhu et al., Science 261: 209-211, 1993). Many of these physical methods can be combined with one another and with viral techniques; enhancement of receptor-mediated DNA uptake can be effected, for example, by combining its use with adenovirus (Curiel et al., Proc. Natl. Acad. Sci. USA 88: 8850-8854, 1991; Cristiano et al., Proc. Natl. Acad. Sci. USA 90: 2122-2126, 1993). Other examples include stem cells such as mesenchymal stem cells, hematopoietic stem cells, cardiac stem cells or neural stem cells, embryonic stem cells that have been engineered to express a sequence of interest.

### Pharmaceutical Compositions

Pharmaceutical compositions of the disclosure can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions may be delivered locally at or in the area of cancer. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

In addition to the active ingredients, these pharmaceutical compositions can contain suitable pharmaceutically acceptable carriers comprising excipients and auxiliaries which facilitate processing of the active compounds into preparations which can be used pharmaceutically.

Pharmaceutical compositions comprising a AOI compound of the present invention in free form or in a pharmaceutically acceptable salt form in association with at least one pharmaceutically acceptable carrier or diluent may be manufactured in a conventional manner by mixing, granulating or coating methods. For example, oral compositions may be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets, together with c) binders, e.g., magnesium aluminum silicate, starch paste, gelatin, tragacanth, methylcellulose, sodium carboxymethylcellulose and/or polyvinylpyrrolidone; and if desired, d) disintegrants, e.g., starches, agar, alginic acid or its sodium salt, or effervescent mixtures; and/or e) absorbents, colorants, flavors and sweeteners. Injectable compositions may be aqueous isotonic solutions or suspensions, and suppositories may be prepared from fatty emulsions or suspensions.

Further, the compounds (e.g. protein or delivery vehicle) for use in the method of the invention can be formulated in a sustained release preparation. For example, the compounds can be formulated with a suitable polymer or hydrophobic material which provides sustained and/or controlled release properties to the active agent compound. As such, the compounds for use the method of the invention can be administered in the form of microparticles for example, by injection or in the form of wafers or discs by implantation.

In additional embodiments, the composition comprises sRNA or miRNA specific for FGD4, an antisense nucleotide specific for FGD4, and/or shRNA specific for FGD4 or a delivery vehicle expressing the preceding AOI compounds. In an alternative embodiment, the composition comprises an antibody specific to frabin.

In another embodiment, administering a therapeutically effective amount of a composition includes a composition comprising: a composition that inhibits the expression or action of FGD4, and a pharmaceutically acceptable excipient.

In further embodiments, the composition includes a FGD4 miRNA, an FGD4 siRNA, an FGD4 shRNA, an antibody specific to FGD4 expression product, and/or an antisense nucleotide specific for FGD4, or delivery vehicles designed for provision of the same. In an alternative embodiment, the composition includes an antibody targeting a cancer cell receptor (e.g. phospholipase 2 or androgen receptor) linked with a AOI.

Many of the embodiments of the subject invention make reference to particular methods of inhibiting expression. The subject invention is not to be limited to any of the particular methods described. One such method includes sRNA (small interfering/short interfering/silencing RNA). SiRNA most often is involved in the RNA interference pathway where it interferes with the expression of a specific gene. In addition to its role in the RNA interference pathway, sRNA also act in RNA interference-related pathways, e.g., as an antiviral mechanism or in shaping the chromatin structure of a genome.

Another method by which to inhibit expression and to inhibit the expression of FGD4 in particular is shRNA. ShRNA (short hairpin or small hairpin RNA) refers to a sequence of RNA that makes a tight hairpin turn and is used to silence gene expression via RNA interference. It uses a vector introduced into cells and a U6 or H1 promoter to ensure that the shRNA is always expressed. The shRNA hairpin structure is cleaved by cellular machinery into sRNA which is then bound to the RNA-induced silencing complex. This complex binds to and cleaves mRNAs which match the siRNA that is bound to it.

FGD4 can also be blocked by subjecting procured cells to an antibody specific to FGD4 expression product (e.g. frabin). An antisense nucleotide may also be used to block or inhibit expression, in particular, the expression of FGD4. Expression may also be inhibited with the use of a morpholino oligomer or phosphorodiamidate morpholino oligomer (PMO). PMOs are an antisense technology used to block access of other molecules to specific sequences within nucleic acid. PMOs are often used as a research tool for reverse genetics, and function by knocking down gene function. This is achieved by preventing cells from making a targeted protein or by modifying splicing of pre-mRNA.

### EXAMPLES

The role of miRNAs in progression of androgen sensitive prostate cancer to CRPC has not been clearly defined. To study this transition, androgen sensitive (AS) LNCaP prostate cancer cells were subjected to androgen deprivation and androgen receptor antagonist Casodex (CDX) until a subset of cells (CDXR) survived. Genome-wide expression profiling of miRNAs identified a subset of miRNAs that are significantly deregulated in these cells. miR-17-92 cluster is one of the groups of miRNAs that are down regulated as the cancer cells progressed towards androgen blockade therapy (ADT) resistance. More than 4 to 24-fold down regulation of these miRNAs were noted in CDXR compared to AS LNCaP cells. The expression status of miR-17-92 cluster in patient tumor tissues was monitored, which showed down regulation of these miRNAs in 64-82% of the tissues tested. Target prediction database searches identified FGD4/Frabin, a novel Rho-GEF as one of the targets of this cluster. Previous studies have shown FGD4 to be involved in filopodia formation and cell migration through interaction with CDC42. Beyond this, little is known about FGD4 function in cancer or whether it is involved in the development of CRPC. Western blot analysis of treated cell lysates confirmed increased expression of FGD4 in AI and CDXR LNCaP cells. Analysis in tissue microarray (267 cores) showed a significant up regulation of FGD4 in advanced prostate cancer tissues including AI specimens. More than 90% of the AI tissues and 88% of tissues with 8-10 Gleason scores showed a median staining intensity between 2-3-fold higher compared to BPH tissues. Ectopic expression of mir-17-92 clusters in AI PC3 cells down regulated FGD4 expression and improved sensitivity of these cells to docetaxel (DTX) treatments.

TABLE 1
Cell lines and treatments

| | | Samples | | |
|---|---|---|---|---|
| | | Cell Line | Treatment | Time point |
| Reference | 0 hr | LNCaP- | FBS-DMT 1 nM | 0 hr |
| | | 104S | | |
| Test samples | 1 wk | LNCaP- | CSFBS | 1 wk |
| | CSFBS | 104S | | |
| | 3 wks | LNCaP- | CSFBS | 3 wks |
| | CSFBS | 104S | | |
| | 1 wk | LNCaP- | CSFBS/5 μM | 1 wk |
| | CDX | 104S | CDX | |
| | 3 wks | LNCaP- | CSFBS/5 μM | 3 wks |
| | CDX | 104S | CDX | |
| Test/Reference | 0 hr | LNCaP- | CSFBS | 0 hr |
| | | 104R1 | | |

MiroRNA Profiling and Identification of Deregulated miRNA Cluster:

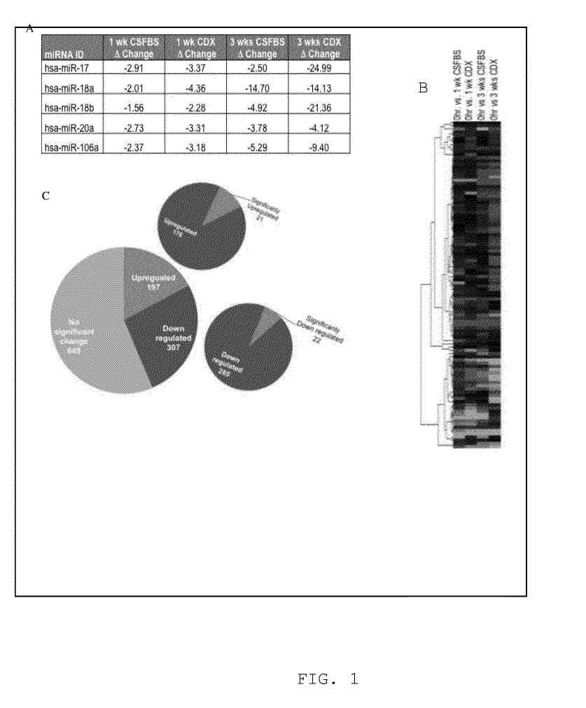

We used genome-wide miRNA array (1113 unique primers System Biosciences) profiling approach to dentify specific miRNAs that compensate for androgen ablation. Androgen-dependent (AD) subline of LNCaP cells LNCaP-104S (-104S) and its androgen independent (AI) derivative LNCaP-104R1 (-104R1) were used for monitoring differential expression of miRNAs upon treatment with casodex (CDX). LNCaP-104S cells are CDX-sensitive, whereas LNCaP-104R1 cells are not despite these cells express androgen receptor (AR) at a basal level higher than LNCaP-104S cells [1]. LNCaP-104S cells require DHT for maintaining their AD status but when treated with CDX for 3 weeks in CSFBS (charcoal-stripped FBS), CDX insensitive colonies develop that are independent of androgen (CDXR). We profiled miRNA expression in −104S cells untreated and at 1 wk and 3 wks of treatment with CDX (5 μM) and CSFBS (Table 1). We also compared miRNA expression in untreated −104R1 cells. The ΔΔCT values were obtained for each sample (treatment condition) using LNCaP-104S untreated as the control. Clustering analyses using log 2 transformed fold change (FC) values of four treatment conditions compared to—104S untreated cells showed a number of down regulated and up regulated miRNAs. Among these miRNAs, the members of the mir-17-92 and its paralogous mir-106a-363 clusters, mir-17, mir-18a, mir-18b, mir-20a and mir-106a showed ˜9-10-fold down regulation (based on the >2.0 FC values with a p value cut off <0.05) upon CDX treatment and androgen blockade from a subset of 43 miRNAs (FIG. 1).

Target Identification:

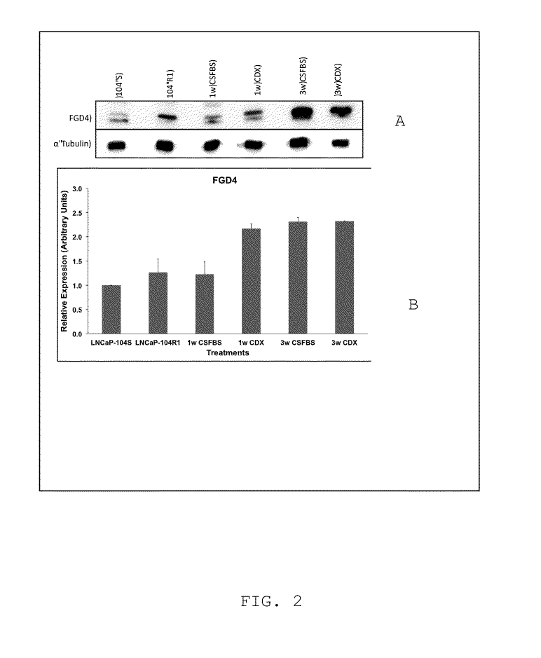

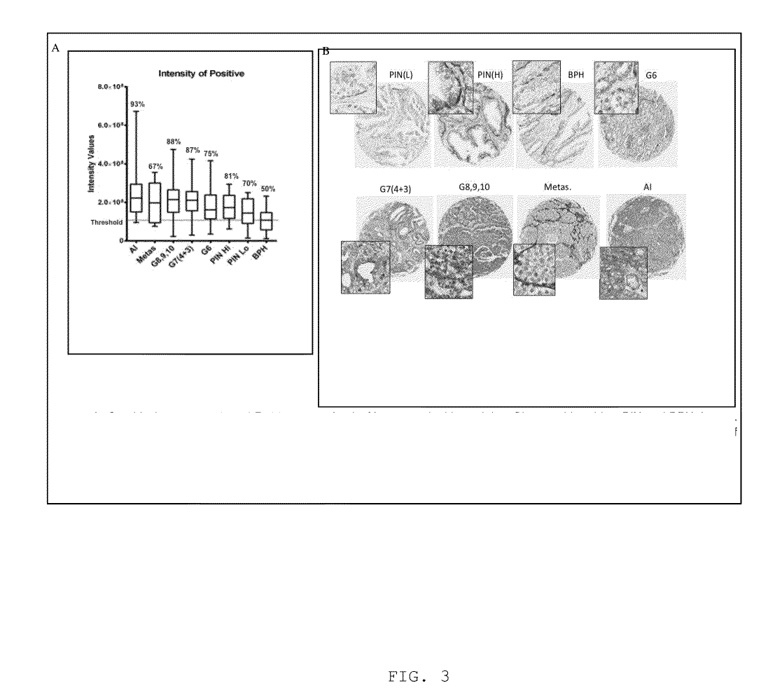

Identification of targets regulated by our candidate miRNAs by MiRDB and TargetScan database search reveled a protein Frabin (FGD4), a GEF that is potentially regulated by mir-17, -21a and -106a and received higher target scores in both searches. Target validation by western blotting using extracts of untreated or treated LNCaP-104S cells and untreated LNCaP-104R1 cells showed >2.0-fold increase in expression of Frabin in CDX treated cells (FIG. 2). Interestingly, LNCaP-104R1 cells do not exhibit significantly higher expression of Frabin compared to the -104S cells, which suggests that the transient up regulation of this protein is facilitating these cells to acquire ADT resistance. Analysis of Frabin expression in clinical samples (TMA of 213 cores) (Table 2) showed a significant up regulation of Frabin in advanced prostate cancer tissues including androgen independent (AI) specimens (FIG. 3). More than 90% of the AI tissues and 88% of tissues with 8-10 Gleason Scores showed a median staining intensity between 2-3-fold higher compared to BPH tissues (FIG. 3A). Our result suggests that Frabin is a novel candidate that may be involved in development of antiandrogen resistance.

TABLE 2
Number and categories of prostate tissues

| Tissue | # of Cores |
|---|---|
| BPH | 23 |
| LGPIN | 24 |
| HGPIN | 15 |
| GS6 | 32 |
| GS7 | 39 |
| GS 8, 9, 10 | 51 |
| M | 10 |
| AI | 19 |

LGPIN: Low grade PIN;
HGPIN: High grade PIN;
GS: Gleason Score;
M: Metastatis;
AI: androgen independent

MiRNA Expression Profiling in Prostate Tumor Tissues:

Expressions of these miRNAs were tested in a small number of prostate tumor tissues surgically removed from patients (Table 3).

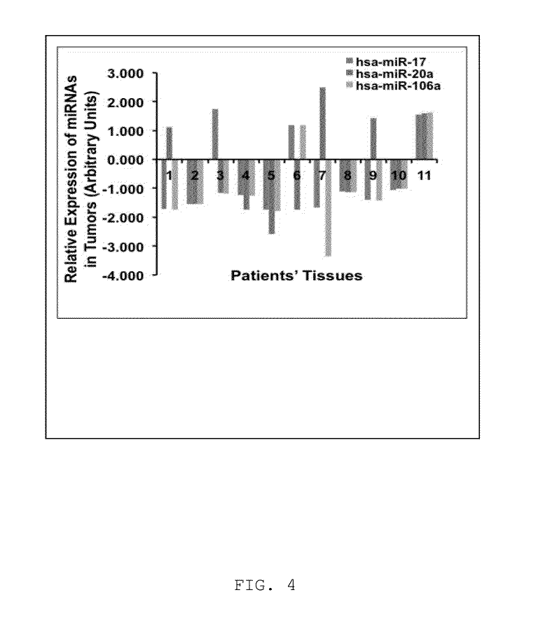

Formalin-fixed paraffin-embedded tissues obtained from Cooperative Human Tissue Network (CHTN, NCI) were macrodissected for the tumor and uninvolved areas and used for miRNA extractions and qRT PCR. Results were analyzed by the ΔΔCT method using matched uninvolved tissues as the controls. Our results showed down regulation of mir-17, mir-20a and mir-106a in the 64-82% of the tissues tested (FIG. 4).

TABLE 3
Pathological reports of the patients

| | | | Surgical | Extra | Seminal | Lymph | | | Risk of |
|---|---|---|---|---|---|---|---|---|---|
| | PSA | Gleason | Margin | capsular | Vesicle | Node | Clinical | Capra | Biochemical/ |
| Patients | level | Score | Status | Extension | Invasion | Invasion | Stage | Score | Recurrance |
| 1 | 23.3 | 3 + 3 | −ve | none | none | −ve | PT2NOMX | 3 | Low |
| 2 | 6.6 | 3 + 4 | −ve | none | none | −ve | PT2ONXMX | 2 | Low |
| 3 | 8.7 | 3 + 4 | −ve | none | none | −ve | PT2ONXMX | 2 | Low |
| 4 | 7.8 | 3 + 4 | −ve | none | none | −ve | PT2ONOMX | 2 | Low |
| 5 | 5.4 | 4 + 3 | −ve | none | invasion | −ve | PT3BNOMX | 4 | Medium |
| 6 | 9.8 | 3 + 4 | +ve | none | none | −ve | PT3AR1NOMX | 4 | Medium |
| 7 | 9.4 | 3 + 4 | +ve | none | none | −ve | PT2ONOMX | 4 | Medium |
| 8 | 5.1 | 3 + 3 | +ve | none | invasion | −ve | PT3BNOMX | 4 | Medium |
| 9 | 6.3 | 3 + 4 | +ve | none | none | −ve | PT3BNOMX(IV) | 4 | Medium |
| 10 | 4.8 | 3 + 4 | +ve | present | invasion | +ve | PT3BR1N1MX | 7 | High |
| 11 | 31 | 4 + 4 | −ve | invasion | invasion | +ve | PT3BN1MX | 11 | High |

Ectopic expression of mir-17-92 cluster in prostate cancer cells:

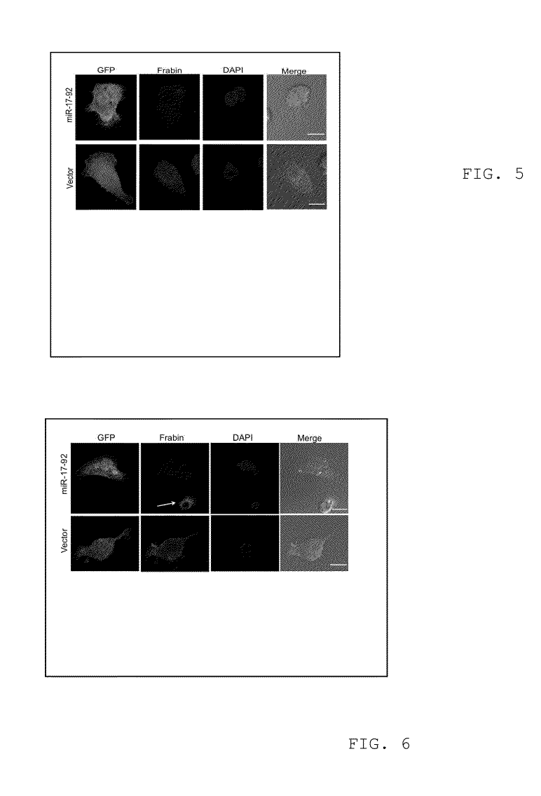

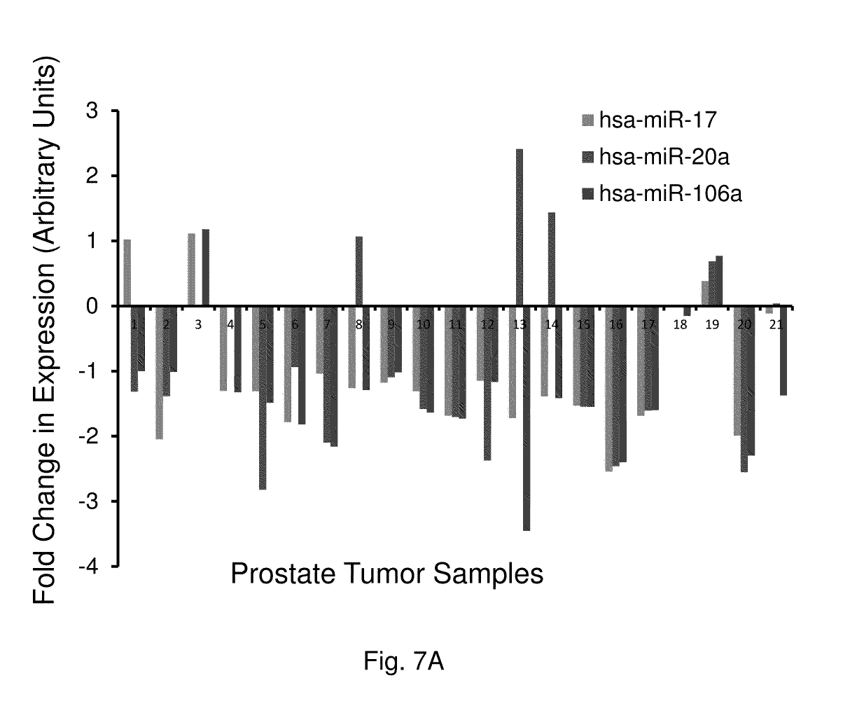

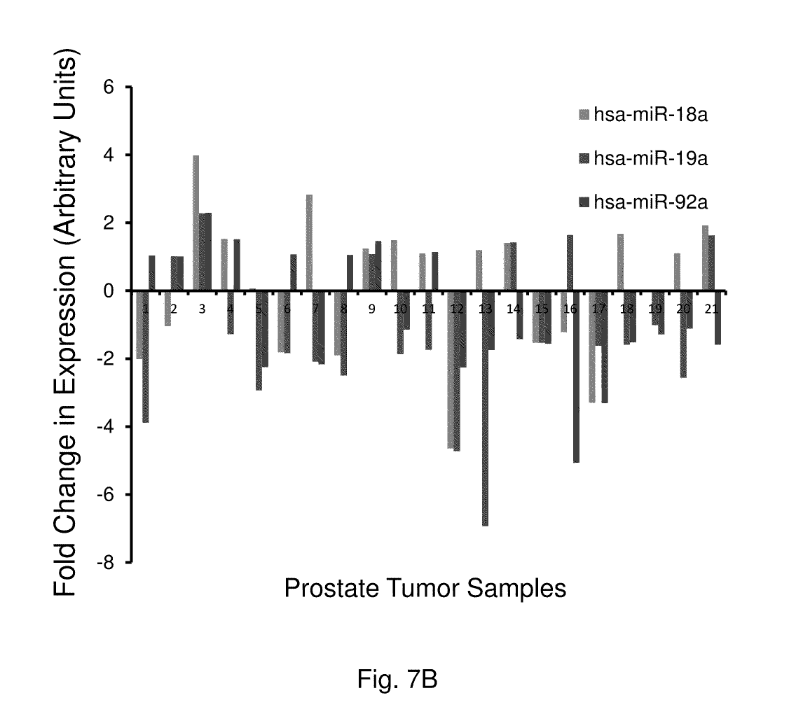

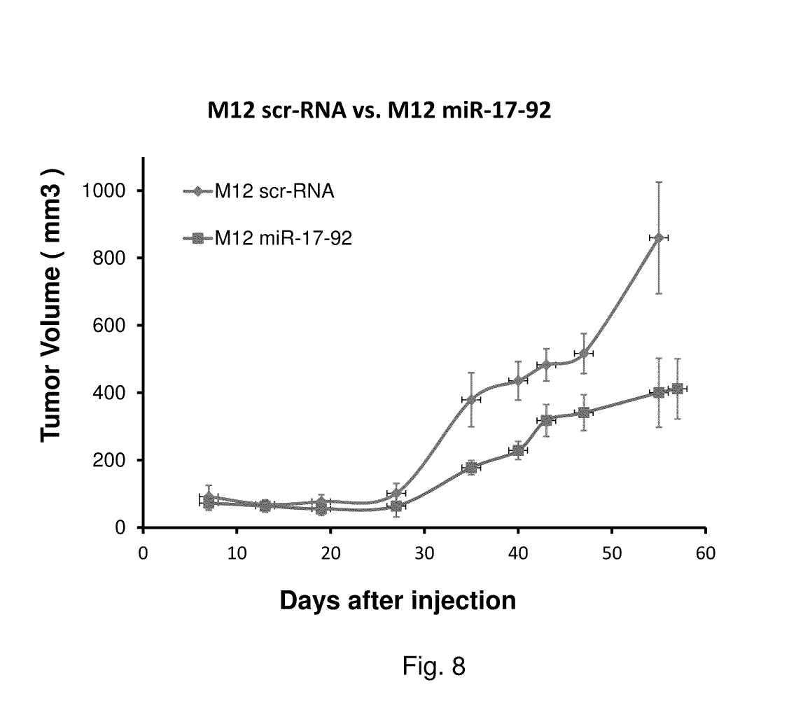

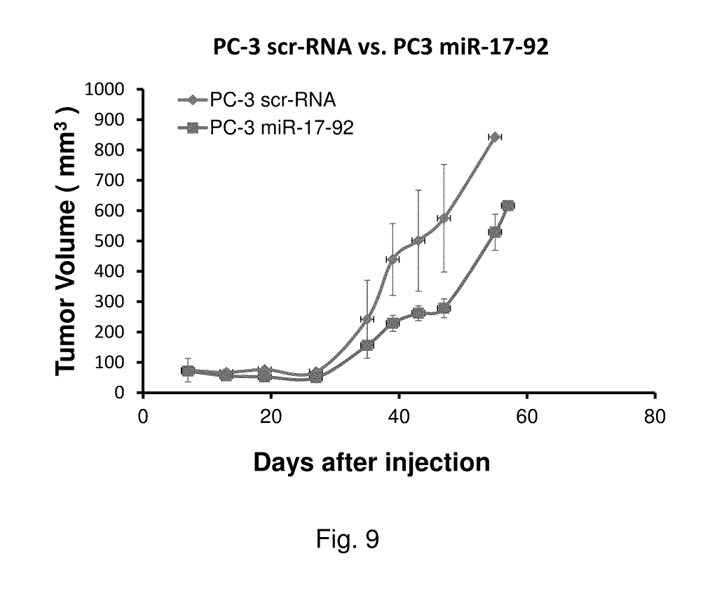

To understand the functional significance of mir-17 and -20a in prostate cancer cells we overexpressed the precursors of mir-17-92 cluster in PC3 cells. Because mir-17, -20a is part of a polycistronic miRNA cluster of six miRNAs (mir-17-92 comprising miR-17, miR-18a, miR-20a, miR-19a, miR-19b-1, and miR-92a-1) processed from a transcript of C13orf25 [2], we chose a lentiviral expression vector of mir-17, -18a, -19a, -20a, -19b-1 and -92a-1 driven by CMV promoter (PMBRH17-92PA-1, System Bioscience) for expression of the GFP tagged 17-92 cluster. Our results showed a significant decrease in expression of Frabin in 17-92 cluster transfected cells (FIG. 5) in AI PC3 cells. Expression of mir-17-92 cluster also improved sensitivity of PC3 cells to DTX that is evident from the appearance of the pre-apoptotic multinucleated giant cells in the miRNA cluster transfected cells (FIG. 6) [3]. Collectively, these studies indicate that an indirect relationship exists between expression of Frabin and miRNAs, mir-17, -20a and -106a and that this miRNA/mRNA axis has been deregulated during treatment with CDX.

### RELATED REFERENCES

1. Kokontis, J. M., et al., Role of androgen receptor in the progression of human prostate tumor cells to androgen independence and insensitivity. Prostate, 2005. 65(4): p. 287-98.
2. Xiao, C., et al., Lymphoproliferative disease and autoimmunity in mice with increased miR-17-92 expression in lymphocytes. Nat Immunol, 2008. 9(4): p. 405-14.
3. Fukuta, K., et al., Induction of multinucleated cells and apoptosis in the PC-3 prostate cancer cell line by low concentrations of polyethylene glycol 1000. Cancer Sci, 2008. 99(5): p. 1055-62.

### General Provisions

Although more than one route can be used to administer a particular compound, a particular route can provide a more immediate and more effective reaction than another route. Accordingly, the described routes of administration are merely exemplary and are in no way limiting.

It should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

While one or more embodiments of the present invention have been shown and described herein, such embodiments are provided by way of example only. Variations, changes and substitutions may be made without departing from the invention herein. Accordingly, it is intended that the invention be limited only by the spirit and scope of the appended claims. The teachings of all references cited herein are incorporated in their entirety to the extent not inconsistent with the teachings herein.

1. A method for treating cancer in a subject, and/or reducing aggressiveness of said cancer, comprising administering to the subject a therapeutically effective amount of a composition that inhibits the expression or action of FGD4 in the subject.

2. The method of claim 1, wherein the composition comprises a viral mediated RNA interfering molecule targeting FGD4.

3. The method of claim 2, wherein the composition comprises a lentivirus comprising at least one of SEQ ID NOs 1-8.

4. The method of claim 1, wherein said composition comprises an agent of interest (AOI) compound that inhibits FGD4 expression in the subject, the AOI being an miRNA targeting FGD4 mRNA.

5. The method of claim 4, wherein the compound comprises an miRNA of SEQ ID NO. 2, 3, 4, 5, 6, or 7, or a combination thereof.

6. The method of claim 4, wherein the compound is administered intravenously or local to the cancer of the subject.

7. A method of reducing androgen blockade insensitivity of prostate cancer in a subject undergoing androgen blockade therapy, said method comprising administering a therapeutically effective amount of a composition comprising an agent of interest (AOI) compound that inhibits the expression or action of FGD4 in the subject.

8. The method of claim 7, wherein the AOI compound is provided in a pharmaceutical composition with a pharmaceutically acceptable carrier.

9. The method of claim 7, wherein the AOI compound comprises FGD4 RNA interfering molecule, antisense molecule, or a delivery vehicle comprising an expressible sequence related thereto.

10. The method of claim 9, wherein said delivery vehicle is a viral vector.

11. The method of claim 10, wherein said viral vector is AAV or lentivirus

12. The method of claim 7, wherein the AOI compound comprises an antibody specific to an expression product of FGD4.

13. A composition comprising an agent of interest (AOI) compound that inhibits expression of FGD4.

14. The composition of claim 13, wherein said AOI comprises FGD4 RNA interfering molecule, antisense molecule, or a delivery vehicle comprising an expressible sequence related thereto.

15. The composition of claim 14, wherein the delivery vehicle is a viral vector.

16. The composition of claim 15, wherein the viral vector is AAV or lentiviral vector comprising at least one sequence of SEQ ID NOs 1-8.

17. The composition of claim 13, wherein said AOI is antibody specific to the expression product of FGD4.

18. The method of claim 10, wherein the viral vector comprises at least one sequence of SEQ ID NOs 1-8.

19. (canceled)

20. (canceled)
